(19) United States
(12) Patent Application Publication
(10) Pub. No.: US 2025/0213633 A1
(43) Pub. Date: Jul. 3, 2025

(54) PHARMACEUTICAL COMPOSITIONS

(71) Applicants: Hisham Hassan Abdelwahab, Abu Dhabi (AE); Mohamed Osman Abdelmonem, Abu Dhabi (AE)

(72) Inventors: Hisham Hassan Abdelwahab, Abu Dhabi (AE); Mohamed Osman Abdelmonem, Abu Dhabi (AE)

(21) Appl. No.: 19/005,881

(22) Filed: Dec. 30, 2024

Related U.S. Application Data
(60) Provisional application No. 63/615,415, filed on Dec. 28, 2023.

(51) Int. Cl. A61K 36/185; A61K 9/48; A61P 31/14

(52) U.S. Cl. CPC A61K 36/185; A61K 9/485; A61K 9/4858; A61K 9/4866; A61P 31/14

(57) ABSTRACT

A method of treating or reducing symptoms of coronavirus disease includes administering a therapeutically effective amount of a pharmaceutical composition to a patient, the pharmaceutical composition including an extract of Balanites aegyptiaca, wherein the pharmaceutical composition is capable of treating or reducing symptoms of coronavirus disease. Pharmaceutical compositions of the present disclosure can be used for treating or reducing symptoms of COVID-19.

### CROSS-REFERENCE TO RELATED APPLICATION

This application claims benefit of U.S. Provisional Application No. 63/615,415, filed on Dec. 28, 2023. U.S. Provisional Application No. 63/615,415 is incorporated herein by reference. A claim of priority is made.

### TECHNICAL FIELD

The subject matter disclosed herein relates to pharmaceutical compositions and more particularly pharmaceutical compositions for diagnosis, prevention, and/or treatment of diseases and conditions. The subject matter disclosed herein further relates to methods for forming such pharmaceutical compositions.

### BACKGROUND

The World Health Organization (WHO) declared the COVID-19 outbreak as a Public Health Emergency of International Concern (PHEIC) on Jan. 30, 2020, and a pandemic was declared on Mar. 11, 2020. COVID-19, or (SARS-COV-2), is a severe acute respiratory syndrome coronavirus, first isolated from patients with respiratory illness in Wuhan. COVID-19 is presumed to be of zoonotic origin. The virus is enveloped with single-stranded RNA genome, and it is genetically one of the coronavirus clusters of genus Betacoronavirus and subgenus Sarbecovirus (lineage B), together with two bat-derived strains. The main presentation of COVID-19 generally includes one or more of fever, sore throat, headache, myalgia, arthralgia, fatigue, sputum production, dry cough, and shortness of breath. While antiviral medications have been developed, these medications remain ineffective in treating coronavirus. Compositions of the present disclosure are capable of effectively treating and/or reducing symptoms of coronavirus.

### SUMMARY

According to one aspect, a method of treating or reducing symptoms of coronavirus disease includes: administering a therapeutically effective amount of a pharmaceutical composition to a patient, the pharmaceutical composition including an extract of Balanites aegyptiaca, wherein the pharmaceutical composition is capable of treating or reducing symptoms of coronavirus disease.

According to another aspect, a method of treating or reducing symptoms of COVID-19 includes administering a therapeutically effective amount of an antiviral pharmaceutical composition to a patient, the antiviral pharmaceutical composition including a pharmaceutical carrier and an extract of Balanites aegyptiaca, wherein the antiviral pharmaceutical composition is capable of inhibiting severe acute respiratory syndrome coronavirus 2 (SARS-COV-2) proliferation.

According to another aspect, a pharmaceutical composition for treatment of coronavirus disease, the composition including: an extract of Balanites aegyptiaca including steroidal Saponins.

### BRIEF DESCRIPTION OF DRAWINGS

This written disclosure describes illustrative embodiments that are non-limiting and non-exhaustive. Reference is made to illustrative embodiments that are depicted in the figures, in which:

FIG. 1 illustrates a method for treating or reducing symptoms of coronavirus disease, according to some embodiments.

FIG. 2 illustrates a method for treating or reducing fibrosis and/or cirrhosis, according to some embodiments.

FIG. 3 illustrates a COVID-19 experimental study design, according to some embodiments.

FIG. 4 illustrates a COVID-19 experimental study design, according to some embodiments.

FIG. 5A illustrates the nationality distribution in the selected COVID-19 study group, according to some embodiments.

FIG. 5B illustrates the gender distribution in the selected COVID-19 study group, according to some embodiments.

FIG. 5C illustrates the age distribution in the selected COVID-19 study group, according to some embodiments.

### DETAILED DESCRIPTION

Pharmaceutical compositions of the present disclosure generally include an extract from one or more species in the Balanites genus and can be utilized for treating and/or reducing symptoms of one or more of coronavirus, fibrosis, and cirrhosis. The extract of one or more species in the Balanites genus include an active pharmaceutical ingredient. The Balanites genus includes Balanites aegyptiaca, Balanites angolensis, Balanites glabra, Balanites maughamii, Balanites pedicellaris, Balanites rotundifolia, Balanites roxburghii, Balanites trifloral, Balanites wilsoniana.

The extract can include an extract from Balanites aegyptiaca. In one example, the extract of Balanites aegyptiaca (also referred to hereafter as “the extract”) is at least one of the active pharmaceutical ingredients (API) in the pharmaceutical composition of the present disclosure. Balanites aegyptiaca is a member of the Zygophyllaceae family and the Balanites genus. Balanites aegyptiaca is common in many areas such as Africa, India, and northern and central Sudan. The fruits of Balanites aegyptiaca may be referred to as “desert date” or “Laloub” fruit. The extract of Balanites aegyptiaca is extracted from one or more materials of a Balanites aegyptiaca species. The extract is generally a concentrated preparation derived from at least a portion of a plant/tree. In one example, the extract of Balanites aegyptiaca includes an extract from Balanites aegyptiaca bark. In another example, the extract of Balanites aegyptiaca includes an extract from one or more of Balanites aegyptiaca root and Balanites aegyptiaca fruit. For example, the extract of the present disclosure may include an extract from Balanites aegyptiaca pulp, seeds, and/or leaves.

The extract can include an aqueous extract obtained from dried Balanites aegyptiaca bark, extracted by boiling. The extract may include linoleic acid. Linoleic acid is precursor of Arachidonic acid (AA) and other unsaturated fatty acids (such as cicosatetraenoic acid, EPA, and docosahexaenoic acid DHA). In one example, these unsaturated fatty acids are capable of inactivating enveloped viruses and inhibiting virus proliferation, such as COVID-19, SARS, and Middle Eastern Respiratory Syndrome (MERS) viruses. The extract may include one or more phenolic compounds. In one example, phenolic compounds include at least one of 2,4-di-tertbutyl-phenol, 2,6-di-tert-butyl-phenol, 3-hydroxy-1-(4-hydroxy-3-methoxyphenyl)-1-propanone, syringic acid, vanillic acid, and coumarins. Importantly, since the extract may include phenolic compounds, pharmaceutical compositions of the present disclosure are capable of antiviral activity.

The extract may include Quercetin and/or Quercetin derivatives. In one example, Quercetin and/or Quercetin derivatives are capable of inhibiting coronavirus proteases, such as the 3cLpro protease. In another example, Quercetin is capable of modulating the cellular unfolded protein response (UPR) of coronavirus sufficient to exhibit antiviral properties. The extract may include a triterpenoid component. For example, the triterpenoid component is capable of reducing airway neutrophilia and reducing concentrations of proinflammatory cytokines and chemokines. This can allow lung tissue to heal, preventing or reducing the chances of lung fibrosis.

The extract may include one or more of furanocoumarin bergapten and dihydrofuranocumarin D-marmesin. In one example, the extract includes one or more of Deltonin and protodeltonin. In another example, the extract includes one or more of alkaloids, metabolites, long-chain aliphatic compounds, sugars, beta-sitosterols, and beta-sitosterol glucosides. Examples of alkaloids in the extract include N-trans-feruloyltramine, N-cis-feruloyltramine, and Trigonelline. Examples of metabolites in the extract include vanillic acid, syringic acid; and 3-hydroxy-1-(4-hydroxy-3-methoxyphenyl)-1-propanone. An example of a long-chain aliphatic compound in the extract is 10-methyl-n-heptacosane. An example of a sugar in the extract is diglucosyldirhamnoside. Examples of beta-sitosterols in the extract include bergapten and marmesin.

The extract may include one or more saponins. Saponins are bioorganic compounds that exhibit triterpenoid or steroidal skeletons that are glycosylated by varying numbers of sugar moieties attached at different positions. Steroidal saponins are further classified into spirostanol, furostanol, and open-chain steroidal saponins. Various spirostanol, furostanol, and open-chain steroidal saponins, can be isolated from fruits, seeds, roots, and stem bark. Examples of spirostanol saponins include Balanitin 1, Balanitin 2, Balanitin 3, Balanitin 4, Balanitin 5, Balanitin 6, Balanitin 7, and Deltonin. The extract can include a more concentrated percentage of saponins compared to the concentration found in the native plant material.

The extract may be in the form of a spray dried extract or a concentrated liquid extract, and the pharmaceutical composition may include a pharmaceutical carrier. The pharmaceutical carrier may be a biocompatible component for transporting the extract. For example, the carrier may be a liquid, such as water, or a solid capsule for enclosing the spray dried extract and/or one or more other components. The solid capsule may be a pharmaceutical carrier known to one of ordinary skill in the art. Therefore, the formulation may include a liquid (syrup) formulation or a solid (capsule) formulation.

In one example, the Balanites aegyptiaca extract is a spray dried extract, and the weight percentage of the spray dried extract in the composition is greater than about 70 wt. %. In another example, the Balanites aegyptiaca extract is a spray dried extract, and the weight percentage of the spray dried extract in the composition is greater than about 80 wt. %. In another example, the Balanites aegyptiaca extract is a spray dried extract, and the weight percentage of the spray dried extract in the composition is greater than about 82 wt. %, greater than about 84 wt. %, greater than about 86 wt. %, greater than about 88 wt. %, greater than about 90 wt. %, or values therebetween. In yet another example, the Balanites aegyptiaca extract is a spray dried extract, and the weight percentage of the spray dried extract in the composition ranges from about 75 wt. % to 95 wt. %.

In one example, the Balanites aegyptiaca extract is a spray dried extract, and the weight/volume percentage of the spray dried extract in the composition is greater than about 70 wt./vol %. In another example, the Balanites aegyptiaca extract is a spray dried extract, and the weight/volume percentage of the spray dried extract in the composition is greater than about 80 wt./vol %. In another example, the Balanites aegyptiaca extract is a spray dried extract, and the weight/volume percentage of the spray dried extract in the composition is greater than about 82 wt./vol %, greater than about 84 wt./vol %, greater than about 86 wt./vol %, greater than about 88 wt./vol %, greater than about 90 wt./vol %, or values therebetween. In yet another example, the Balanites aegyptiaca extract is a spray dried extract, and the weight/volume percentage of the spray dried extract in the composition ranges from about 75 wt./vol % to 95 wt./vol %.

In one example, the Balanites aegyptiaca extract is a concentrated liquid extract, and the weight/volume percentage of the concentrated liquid extract in the composition ranges from about 2 wt./vol % to about 70 wt./vol %. In another example, the Balanites aegyptiaca extract is a concentrated liquid extract, and the weight/volume percentage of the concentrated liquid extract in the composition ranges from about 5 wt./vol % to about 60 wt./vol %. In another example, the Balanites aegyptiaca extract is a concentrated liquid extract, and the weight/volume percentage of the concentrated liquid extract in the composition ranges from about 5 wt./vol % to about 40 wt./vol %, or about 5 wt./vol % to about 20 wt./vol %. In yet another example, the Balanites aegyptiaca extract is a concentrated liquid extract, and the weight/volume percentage of the concentrated liquid extract in the composition is greater than about 5 wt./vol %, greater than about 7 wt./vol %, greater than about 9 wt./vol %, or values therebetween. In one non-limiting example, the concentrated liquid extract includes 25% of the amount of active ingredient in the spray dried extract.

The concentrated liquid extract may include about 10 wt. % to about 100 wt. % of Balanites aegyptiaca extract. In one example, the concentrated liquid extract includes between about 15 wt. % to about 50 wt. % of Balanites aegyptiaca extract. In another example, the concentrated liquid extract includes between about 15 wt. % to about 40 wt. % of Balanites aegyptiaca extract. In yet another example, the concentrated liquid extract includes between about 20 wt. % to about 30 wt. % of Balanites aegyptiaca extract.

In one example, the pharmaceutical composition includes between about 50 mg and about 400 mg of Balanites aegyptiaca extract. In another example, the pharmaceutical composition includes between about 150 mg and about 400 mg of Balanites aegyptiaca extract. In another example, the pharmaceutical composition includes between about 200 mg and about 300 mg of Balanites aegyptiaca extract. In yet another example, the pharmaceutical composition includes between about 225 mg and about 275 mg of Balanites aegyptiaca extract. For example, the pharmaceutical composition may include about 250 mg of Balanites aegyptiaca extract. The pharmaceutical composition may include greater than about 150 mg, about 200 mg, or about 250 mg of Balanites aegyptiaca extract. In one example, a pharmaceutical composition capsule includes about 250 mg of Balanites aegyptiaca extract, and a liquid formulation includes about 250 mg of Balanites aegyptiaca extract per about 10 mL. Amounts of the Balanites aegyptiaca extract of the present paragraph may be therapeutically effective amounts.

Therapeutically effective amounts can be utilized in one or more doses to treat and/or reduce symptoms of coronavirus. Treating or reducing symptoms of coronavirus may include reducing days of hospitalization and/or recovery time, reducing the average number of hours or days until a negative PCR test, and/or reducing the severity of any of the symptoms of the present disclosure.

The pharmaceutical composition may include an admixture with one or more excipients. The pharmaceutical composition may include one or more antimicrobial agents. For example, a liquid formulation of the present disclosure may include at least one antimicrobial agent. Examples of suitable antimicrobial agents include methyl paraben, propyl paraben, and potassium sorbate. These antimicrobial agents may act as a preservative for the formulation. The weight/volume percentage of antimicrobial agent(s) in the pharmaceutical composition may range from about 0.1 wt./vol % to about 10 wt./vol %. For example, the weight/volume percentage of antimicrobial agent(s) in the pharmaceutical composition may range from about 0.1 wt./vol % to about 1 wt./vol %.

The pharmaceutical composition may further include one or more viscosity increasing agents. For example, a liquid formulation of the present disclosure may include at least one viscosity increasing agent. An example of a suitable viscosity increasing agent is Acacia gum. In one example, the weight/volume percentage of viscosity increasing agent(s) in the pharmaceutical composition may range from about 2 wt./vol % to about 15 wt./vol %. In another example, the weight/volume percentage of viscosity increasing agent(s) in the pharmaceutical composition may range from about 6 wt./vol % to about 10 wt./vol %. For example, the weight/volume percentage of viscosity increasing agent(s) in the pharmaceutical composition may be about 8 wt./vol %.

The pharmaceutical composition may further include one or more antifoaming agents. For example, a liquid formulation of the present disclosure may include at least one antifoaming agent. The antifoaming agent may be added to prevent or counter foam generation in the liquid formulation. An example of a suitable antifoaming agent is Simethicone. In one example, the weight/volume percentage of antifoaming agent(s) in the pharmaceutical composition may range from about 0.0001 wt./vol % to about 1 wt./vol %. In another example, the weight/volume percentage of antifoaming agent(s) in the pharmaceutical composition may range from about 0.01 wt./vol % to about 1 wt./vol %. For example, the weight/volume percentage of antifoaming agent(s) in the pharmaceutical composition may be about 0.02 wt./vol %.

The pharmaceutical composition may further include one or more sweetening agents. For example, a liquid formulation of the present disclosure may include at least one sweetening agent. The sweetening agent(s) may be added to increase the sweetness of a liquid formulation and may make the formulation more palatable. Examples of a suitable sweetening agents include sugar and liquid glucose. In one example, the weight/volume percentage of sweetening agent(s) in the pharmaceutical composition may range from about 2 wt./vol % to about 30 wt./vol %. In another example, the weight/volume percentage of sweetening agent(s) in the pharmaceutical composition may range from about 7 wt./vol % to about 20 wt./vol %. For example, the weight/volume percentage of sweetening agent(s) in the pharmaceutical composition may be about 15 wt./vol %.

The pharmaceutical composition may include one or more flavoring agents, such as a cherry flavoring agent or a pineapple flavoring agent. For example, a liquid formulation of the present disclosure may include at least one flavoring agent. The flavoring agent may be added to make the pharmaceutical composition more palatable. In one example, the weight/volume percentage of flavoring agent(s) in the pharmaceutical composition may range from about 0.01 wt./vol % to about 5 wt./vol %. In another example, the weight/volume percentage of flavoring agent(s) in the pharmaceutical composition may range from about 0.01 wt./vol % to about 1 wt./vol %. The remainder of the liquid pharmaceutical composition may include one or more solvents, such as water.

The pharmaceutical composition may include one or more disinfectants, alkaline compounds, adsorbents, and lubricants. For example, a capsule or solid formulation may include at least one of disinfectants, antimicrobial agents, alkaline compounds, adsorbents, and lubricants. Disinfectants may also act as a suitable binder for oral tablets, and an example of a suitable disinfectant is pregelatinized starch. The weight percentage of disinfectant(s) in the pharmaceutical composition may range from about 1 wt. % to about 10 wt. %. For example, the weight percentage of disinfectant(s) in the pharmaceutical composition may range from about 2 wt. % to about 4 wt. %. The weight/volume percentage of disinfectant(s) in the pharmaceutical composition may range from about 1 wt./vol % to about 10 wt./vol %. Alkaline compounds may increase solubility, and an example of an alkaline compound is magnesium oxide. The weight percentage of alkaline compound(s) in the pharmaceutical composition may range from about 0.2 wt. % to about 3 wt. %. For example, the weight percentage of alkaline compound(s) in the pharmaceutical composition may range from about 1 wt. % to about 1.75 wt. %. The weight/volume percentage of alkaline compound(s) in the pharmaceutical composition may range from about 0.2 wt./vol % to about 3 wt./vol %.

Adsorbents may be added to the pharmaceutical composition to enhance dissolution by increasing the rate of disintegration. An example of an adsorbent is microcrystalline cellulose. The weight percentage of adsorbent(s) in the pharmaceutical composition may range from about 0.5 wt. % to about 5 wt. %. For example, the weight percentage of adsorbent(s) in the pharmaceutical composition may range from about 2.5 wt. % to about 3.5 wt. %. The weight/volume percentage of adsorbent(s) in the pharmaceutical composition may range from about 0.5 wt./vol % to about 5 wt./vol %. Additionally, or alternatively, lubricants may be added to improve flow properties of a solid formulation. An example of a suitable lubricant is talcum powder. The weight percentage of lubricant(s) in the pharmaceutical composition may range from about 0.5 wt. % to about 5 wt. %. For example, the weight percentage of lubricant(s) in the pharmaceutical composition may range from about 1 wt. % to about 3 wt. %. The weight/volume percentage of lubricant(s) in the pharmaceutical composition may range from about 0.5 wt./vol % to about 5 wt./vol %. A solid formulation of the present disclosure may include between 0.1 wt. % and 3 wt. %, between 0.1 wt. % and 2 wt. %, or between 0.1 wt. % and 1 wt. % of antimicrobial agents of the present disclosure. A solid formulation of the present disclosure may include antimicrobial agents ranging from 0.1 wt./vol % to 3 wt./vol %.

The pharmaceutical composition can be prepared by techniques such as direct compression, wet granulation, or dry granulation. The compositions may be in form of powder and may be filled in capsules or may be compressed into tablets. In one example, the pharmaceutical composition may be administered for at least 3 days, for at least 7 days, for least 10 days, or for at least 14 days. The treatment period may be determined based on the severity of symptoms. The pharmaceutical composition may be in a tablet form sufficient for immediate or extended release.

Pharmaceutical compositions of the present disclosure may be used for treating coronavirus, such as SARS coronavirus and COVID-19. The COVID-19 virus is enveloped with single-stranded RNA genome. The main presentation of the COVID-19 disease includes fever, sore throat, headache, myalgia or arthralgia, fatigue, sputum production, dry cough, and shortness of breath. COVID-19 testing can identify the virus and includes methods that detect the presence of the virus itself (RT-PCR and isothermal nucleic acid amplification of throat and nasopharyngeal samples) and those that detect antibodies produced in response to an infection. Chest CT scans can be used to diagnose COVID-19, where the scan manifests with abnormalities, even in asymptomatic patients. Typical features on CT scans initially include bilateral multilobar ground-glass opacities with a peripheral, asymmetric, and posterior distribution.

Several available antiviral medications have been tested for treatment of COVID-19, including Interferon Beta, lopinavir/ritonavir, remdesivir, chloroquine, and hydroxychloroquine, but none of these have been effective. Currently, there is no approved therapy or strong scientific evidence regarding any therapy for the treatment of COVID-19. Importantly, pharmaceutical compositions of the present disclosure are capable of effectively treating and/or reducing symptoms of coronavirus. Further, these pharmaceutical compositions of the present disclosure can improve patient health for patients suffering from coronavirus related pneumonia and shorten the patient's admission period.

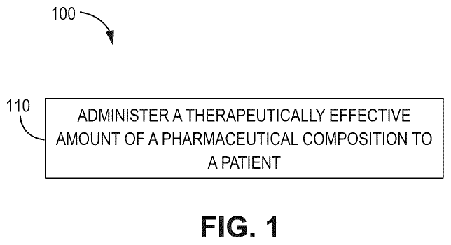

FIG. 1 illustrates method 100 of treating or reducing symptoms of coronavirus disease, according to some embodiments. Method 100 includes the following step:

STEP 110, ADMINISTER A THERAPEUTICALLY EFFECTIVE AMOUNT OF A PHARMACEUTICAL COMPOSITION TO A PATIENT, THE PHARMACEUTICAL COMPOSITION INCLUDING AN EXTRACT OF BALANITES AEGYPTIACA, WHEREIN THE PHARMACEUTICAL COMPOSITION IS CAPABLE OF TREATING OR REDUCING SYMPTOMS OF CORONAVIRUS DISEASE, includes administering a therapeutically effective amount of the pharmaceutical composition of the present disclosure. Administering may include oral administration of a capsule or liquid formulation, or other administration techniques known to one of ordinary skill in the art. The patient generally includes a human patient. The therapeutically effective amount may be administered one or more times per day, such as two times, three times, or four times.

A therapeutically effective amount may be sufficient to reduce symptoms of coronavirus disease when administered. Symptoms of coronavirus include one or more of fever, sore throat, headache, myalgia or arthralgia, fatigue, sputum production, dry cough, and shortness of breath. Treating or reducing symptoms of coronavirus may include reducing days of hospitalization and/or recovery time, reducing the average number of days until a negative PCR test, and/or reducing the severity of any of the symptoms of the present disclosure. Coronavirus disease may include any disease of the present disclosure, such as COVID-19 and MERS.

In one example, a therapeutically effective amount of the pharmaceutical composition includes between about 50 mg and about 500 mg of Balanites aegyptiaca extract. In another example, a therapeutically effective amount of the pharmaceutical composition includes between about 150 mg and about 500 mg of Balanites aegyptiaca extract. In another example, a therapeutically effective amount of the pharmaceutical composition includes between about 200 mg and about 500 mg of Balanites aegyptiaca extract. In yet another example, a therapeutically effective amount of the pharmaceutical composition includes between about 200 mg and about 300 mg of Balanites aegyptiaca extract. For example, a therapeutically effective amount of the pharmaceutical composition may include about 250 mg of Balanites aegyptiaca extract. A therapeutically effective amount of the pharmaceutical composition may include greater than about 150 mg, greater than about 200 mg, or greater than about 250 mg of Balanites aegyptiaca extract.

In one example, the therapeutically effective amount of the pharmaceutical composition is administered once every 2 hours to 12 hours. In another example, the therapeutically effective amount of the pharmaceutical composition is administered once every 3 hours to 6 hours. For example, the therapeutically effective amount of the pharmaceutical composition may be administered once every about 4 hours. In yet another example, about 5 mL to 35 mL, about 8 mL-12 mL, or about 10 mL of the pharmaceutical composition may be administered once every 2-8 hours, every 3-6 hours, or every 4 hours. In yet another example, about 200 mg to about 300 mg of the pharmaceutical composition may be administered once every 2-8 hours, every 3-6 hours, or every 4 hours. In one example, 1000 mg to 1800 mg, or 1200 mg to 1600 mg of Balanites aegyptiaca extract is administered per day. The therapeutically effect amount and administration timing of the present disclosure is capable of shortening coronavirus recovery time when administered. Importantly, the pharmaceutical composition is capable of modulating the cellular unfolded protein response (UPR) of viruses when administered. Further, the pharmaceutical composition is capable of inactivating enveloped viruses and inhibiting virus proliferation when administered.

Methods of the present disclosure include ADMINISTERING A THERAPEUTICALLY EFFECTIVE AMOUNT OF AN ANTIVIRAL PHARMACEUTICAL COMPOSITION, THE ANTIVIRAL PHARMACEUTICAL COMPOSITION INCLUDING A PHARMACEUTICAL CARRIER AND AN EXTRACT OF BALANITES AEGYPTIACA, WHEREIN THE ANTIVIRAL PHARMACEUTICAL COMPOSITION IS CAPABLE OF INHIBITING SEVERE ACUTE RESPIRATORY SYNDROME CORONA VIRUS 2 (SARS-COV-2) PROLIFERATION, includes administering a therapeutically effective amount (as described in the present disclosure) of the pharmaceutical composition of the present disclosure. Administering this therapeutically effective amount is sufficient to inhibit severe acute respiratory syndrome coronavirus 2 (SARS-COV-2) proliferation. In one example, virus proliferation includes gene replication inside cells and transmission to new target cells. Accordingly, the pharmaceutical composition is capable of reducing or preventing virus replication and transmission to new target cells. The pharmaceutical composition can inhibit virus proliferation by inactivating enveloped viruses, and the pharmaceutical composition is capable of modulating the cellular unfolded protein response (UPR) of viruses when the antiviral pharmaceutical composition is administered.

While several antiviral medications have been tested for treatment of COVID-19, none have proven to be effective. Since pharmaceutical compositions of the present disclosure may include an extract of Balanites aegyptiaca including steroidal Saponins, the composition may exhibit antiviral activity. This composition is capable of modulating the cellular unfolded protein response (UPR) of coronavirus sufficient to exhibit these antiviral properties. Importantly, the composition is capable of inactivating enveloped viruses and inhibiting virus proliferation, such as COVID-19, SARs, and Middle Eastern Respiratory Syndrome (MERS) viruses.

Compositions of the present disclosure may exhibit antifibrotic effect and are capable of treating fibrosis and/or cirrhosis. Fibrosis includes thickening or scarring of tissue. One example of fibrosis includes liver fibrosis, where the liver may include an elevated amount of scar tissue. Chronic liver fibrosis is an ominous disease that can lead to liver cirrhosis, hepatocellular carcinoma (HCC), and death. Cirrhosis includes much more severe and serious scarring of the liver compared to fibrosis, and cirrhosis prevents the liver from functioning properly. While there has been an absence of reliable therapy and treatment for fibrosis and cirrhosis, the pharmaceutical compositions of the present disclosure can be used to treat one or more of fibrosis and cirrhosis.

In one example, the antifibrotic effect of the pharmaceutical composition is exerted by the ability to bind and regulate the function of a Lysyl oxidase like enzyme, an enzyme responsible for cross linking of collagen fibers. This enzyme is Copper dependent, a metal which is abundant in fibroid. In another example, the pharmaceutical composition may reduce fibrosis through a calcium dependent mechanism, since the pharmaceutical composition has a high ability to bind Ca⁺², which is responsible for activation of the reaction that converts unstable fibrin to hardened, contracted stable fibrin.

Not only are pharmaceutical compositions of the present disclosure capable of preventing liver fibrosis, but these pharmaceutical compositions are also capable of at least partially reversing liver fibrosis and/or liver cirrhosis, even at the latest stages. In one example, the stages of liver fibrosis are as follows: F0-no fibrosis; F1-portal fibrosis without septa; F2-portal fibrosis with infrequent septa; F3-numerous fibrous septa but no cirrhosis; F4-cirrhosis. These stages may be referred to as Metavir or Batts-Ludwig stages. In one example, the pharmaceutical composition can reverse liver fibrosis from Stage F4 to Stage F3, Stage F4 to Stage F3, Stage F3 to Stage F2, Stage F2 to Stage F1, and/or Stage F2 to F0. Importantly, the pharmaceutical composition is capable of regressing stage 4 liver fibrosis when administered. For example, pharmaceutical compositions of the present disclosure can reverse liver fibrosis from Stage F4 to Stage F1 when administered to a patient.

The pharmaceutical composition is capable of reducing liver elasticity kPa values by over 20% when administered. In one example, the pharmaceutical composition is capable of reducing liver elasticity kPa values by over 30% when administered. Importantly, treatment with the present composition may be completed in 6 months or less, compared to a traditional timeline of 5-7 years with other pharmaceutical compositions. In one example, patients with Stage F2 or F3 fibrosis only require about 2 months of treatment with the present composition. Further, this pharmaceutical composition is capable of reducing and/or removing fat deposits in and/or around the liver, sometimes referred to as fatty liver. Since the pharmaceutical composition may include the saponins Balanitin 1, Balanitin 2, and Balanitin 3, the pharmaceutical composition can lower serum lipids and reverse fatty liver.

An additional example of fibrosis is myocardial fibrosis. In one example, myocardial fibrosis is the expansion of the cardiac interstitium through deposition of extracellular matrix proteins. Accordingly, myocardial fibrosis may include scarring of heart muscles due to a chronic heart injury. This can cause symptoms such as shortness of breath, fatigue, and weakness. While myocardial fibrosis may partially and naturally repair over time, this process may take extensive time (such as many years). In one example, administering the pharmaceutical compositions of the present disclosure at least partially treats myocardial fibrosis. Treating may include reducing recovery time, reducing scar tissue, reducing fibrosis symptoms, and/or otherwise improving organ function.

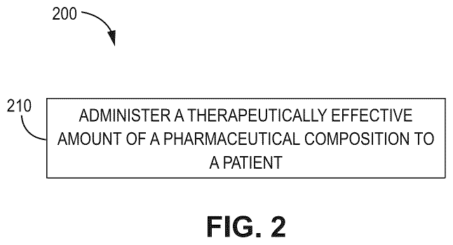

FIG. 2 illustrates method 200 of treating or reducing fibrosis and/or cirrhosis, according to some embodiments. Method 200 includes the following step:

STEP 210, ADMINISTER A THERAPEUTICALLY EFFECTIVE AMOUNT OF A PHARMACEUTICAL COMPOSITION TO A PATIENT, THE PHARMACEUTICAL COMPOSITION INCLUDING A PHARMACEUTICAL CARRIER AND AN EXTRACT OF BALANITES AEGYPTIACA, WHEREIN THE PHARMACEUTICAL COMPOSITION IS CAPABLE OF REDUCING AT LEAST ONE OF FIBROSIS AND CIRRHOSIS WHEN ADMINISTERED, includes administering a therapeutically effective amount of the pharmaceutical composition of the present disclosure. Liquid and solid formulations of the present disclosure may be utilized. The therapeutically effective amount may be administered two or more, or three or more times per day. For example, the therapeutically effective amount may be administered three times per day.

A therapeutically effective amount may be sufficient to treat or reduce symptoms of fibrosis and/or cirrhosis. As stated, examples of fibrosis include liver fibrosis and myocardial fibrosis. Not only are pharmaceutical compositions of the present disclosure capable of preventing liver fibrosis, but these pharmaceutical compositions are also capable of at least partially reversing liver fibrosis and/or liver cirrhosis, even at the latest stages. In one example, treating fibrosis includes reducing the liver fibrosis stage when administered. In another example, treating fibrosis includes reducing liver elasticity kPa values by over 30% when administered. In another example, treating fibrosis includes reducing fatty liver deposits.

In one example, a therapeutically effective amount of the pharmaceutical composition includes between about 50 mg and about 500 mg of Balanites aegyptiaca extract. In another example, a therapeutically effective amount of the pharmaceutical composition includes between about 150 mg and about 500 mg of Balanites aegyptiaca extract. In another example, a therapeutically effective amount of the pharmaceutical composition includes between about 200 mg and about 500 mg of Balanites aegyptiaca extract. In yet another example, a therapeutically effective amount of the pharmaceutical composition includes between about 225 mg and about 300 mg of Balanites aegyptiaca extract. For example, a therapeutically effective amount of the pharmaceutical composition may include about 250 mg of Balanites aegyptiaca extract. A therapeutically effective amount of the pharmaceutical composition may include greater than about 150 mg, greater than about 200 mg, or greater than about 250 mg of Balanites aegyptiaca extract.

Historically, the typical treatment timeline for liver cirrhosis was 5-7 years with conventional pharmaceuticals. The pharmaceutical compositions of the present disclosure may be used to treat liver cirrhosis in as little as a few months. In one example, patients with Stage F2 or F3 fibrosis only require about 2 months of treatment. The pharmaceutical composition is also capable of reducing and/or removing fat deposits in and/or around the liver, sometimes referred to as fatty liver. Further, this pharmaceutical composition may be used to treat myocardial fibrosis.

### Example 1-Pharmaceutical Compositions

One suitable pharmaceutical composition of the present disclosure, Composition A, was formulated according to the formulations in Tables 1 and 2. Table 1 shows the Composition A capsule (solid) formulation, while Table 2 shows the Composition A syrup (liquid) formulation.

TABLE 1
Composition A Capsule Formulation.

| Ingredients | Specification | Qty. mg/caps | W/V % |
|---|---|---|---|
| Spry dried extract | BP | 250 | 89.29 |
| Pregelatinized | BP | 10.88 | 3.89 |
| starch | | | |
| Mg oxide | BP | 04.20 | 01.50 |
| Methyl parben | BP | 0.51 | 0.18 |
| Propyl Parben | BP | 0.11 | 0.04 |
| Microcrystalline | BP | 08.70 | 03.10 |
| cellulose 101 | | | |
| Talcum powder | BP | 05.60 | 02.00 |
| Isopropyl Alcohol | BP | QS | |
| Powder weight | | 280 mg | |

TABLE 2
Composition A Syrup Formulation.

| | Specifi- | Qty. in | Qty. in | Qty. in | w/v |
|---|---|---|---|---|---|
| Ingredients | cation | mg./5 ml | g/bottle | kg/batch | % |
| Concentrated | I.H | 500 [Eq. | 10 [Liquid | 50 [Liquid | 10 |
| Extract | | to 125 mg | Extract Eq. | Extract Eq. | |
| (Liquid) | | Of solid | to 2.50 | to 25 kg | |
| 25% | | Extract/5 | g of Solid | of Solid | |
| | | ml.] | Extract] | Extract] | |
| Methyl Paraben | B.P. | 10.00 | 0.200 | 1 | 0.20 |
| Propyl Paraben | B.P. | 1.00 | 0.020 | 0.10 | 0.02 |
| Potassium | B.P. | 7.50 | 0.150 | 0.75 | 0.15 |
| Sorbate | | | | | |
| Acacia Gum | B.P. | 400 | 8.000 | 40 | 8 |
| Simethicone | B.P. | 1.00 | 0.020 | 0.1 | 0.02 |
| Sugar | B.P. | 500.00 | 10.000 | 50 | 10 |
| Liquid glucose | B.P. | 250.00 | 5.00 | 25 | 5 |
| Cherry flavor | I.H. | 5.00 | 0.10 | 0.50 | 0.10 |
| Pineapple | I.H. | 5.00 | 0.10 | 0.50 | 0.10 |
| flavor | | | | | |
| Purified water | B.P. | 5 ml | 100 ml | 500 lit | |
| up to . . . | | | | | |

Composition A contains Linoleic acid, a precursor of Arachidonic acid, and other unsaturated fatty acids (such as eicosatetraenoic acid, EPA, and docosahexaenoic acid (DHA)). These can inactivate enveloped viruses and inhibit their proliferation, including COVID-19, SARS, and MERS viruses. Composition A also contains phenolic compounds such as 2,4-di-tertbutyl-phenol, 2,6-di-tert-butyl-phenol (seed), 3-hydroxy-1-(4-hydroxy-3-methoxyphenyl)-1-propanone, syringic acid, vanillic acid, and coumarins. Examples of coumarins include Bergapten and Marmesin.

Composition A contains quercetin and its derivatives which inhibit the coronavirus proteases, such as 3CLpro. Further, quercetin is capable of inhibiting the Middle Eastern Respiratory Syndrome coronavirus (MERS-COV) 3CLpro protease. In addition, quercetin modulates the cellular unfolded protein response (UPR) of coronaviruses. The triterpenoid component of Composition A reduced airway neutrophilia, reducing concentrations of proinflammatory cytokines and chemokines. This gives lung tissue a chance to heal and prevents lung fibrosis. Composition A components underwent a comprehensive toxicological evaluation in mice, rabbits, and Bovan chicks. Toxicity studies covered acute toxicity, lethal dose 50, and subchronic toxicity (l8). The LD50 in mice orally was 5440 mg freeze-dried extract/kg and 1320 mg freeze-dried extract/kg.

### Example 2-Pharmaceutical Compositions for Treating COVID-19

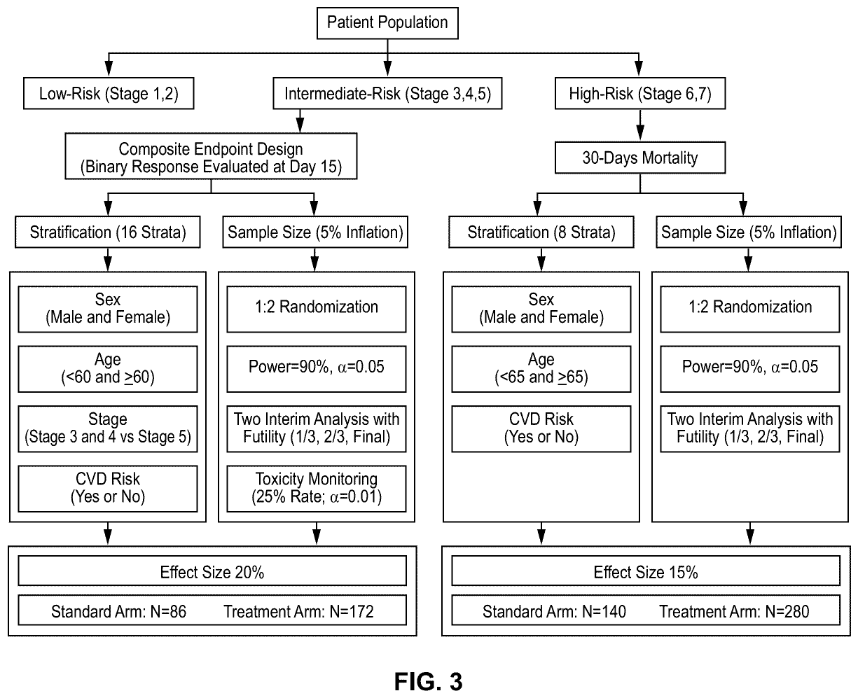

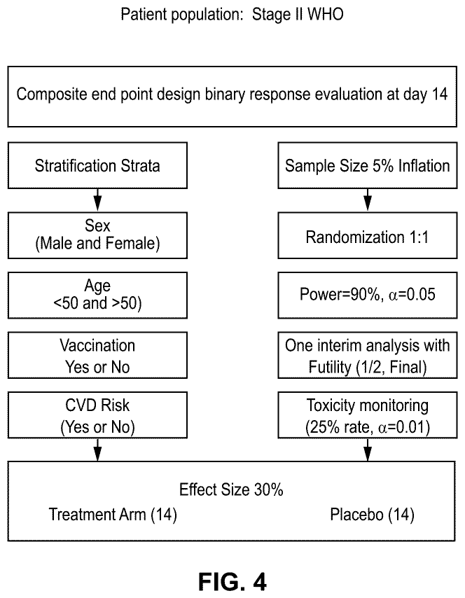

FIG. 3 illustrates a COVID-19 experimental study design, according to some embodiments. FIG. 3 illustrates the trial design for using Composition A to treat COVID-19. Accordingly, the trial was designed for randomization and controlled interventional, double-blind, with intention to treat. FIG. 4 illustrates the COVID-19 experimental study design, according to some embodiments. The initial trial design was for a low-risk group, with composite endpoint design binary response evaluation on day 14. The inclusion criteria for the study were as follows:
1-Admitted to a hotel or hospital with symptoms suggestive of COVID-19 infection and on standard treatment as per MOH protocol.
2-Patient (or legally authorized representative) provided informed consent prior to initiation of any study procedures.
3-Patient (or legally authorized representative) understood and complied with planned study procedures.
4-Male and non-pregnant female adult 18 years of age or above at time of enrollment.
5-Had laboratory-confirmed COVID-19 infection as determined by polymerase chain reaction (PCR) or other commercial or public health assay in any specimen, as documented by a PCR positive in sample collected <72 hours prior to randomization. The diagnostic criteria were based on guidance from the World Health Organization guidelines, in which confirmed cases of COVID-19 infection were those confirmed by molecular tests with viral sequencing or real-time quantitative PCR (RT-PCR) of swab samples obtained from nasopharyngeal, bronchoalveolar lavage, nasopharyngeal tracheal aspirate.
6-Agreed to not participate in another clinical trial for the treatment of COVID-19 or COVID-19 through Day 29.

The exclusion criteria for the study were as follows:
1-Alanine Transaminase (ALT) or Aspartate Transaminase (AST)>5 times the upper limit of normal.
2-Anticipated discharge from the hospital or transfer to another hospital which is not a study site within 72 hours.
3-Cases with severe diarrhea.
4-Allergic to Composition A.
5-Pregnant and lactating women.

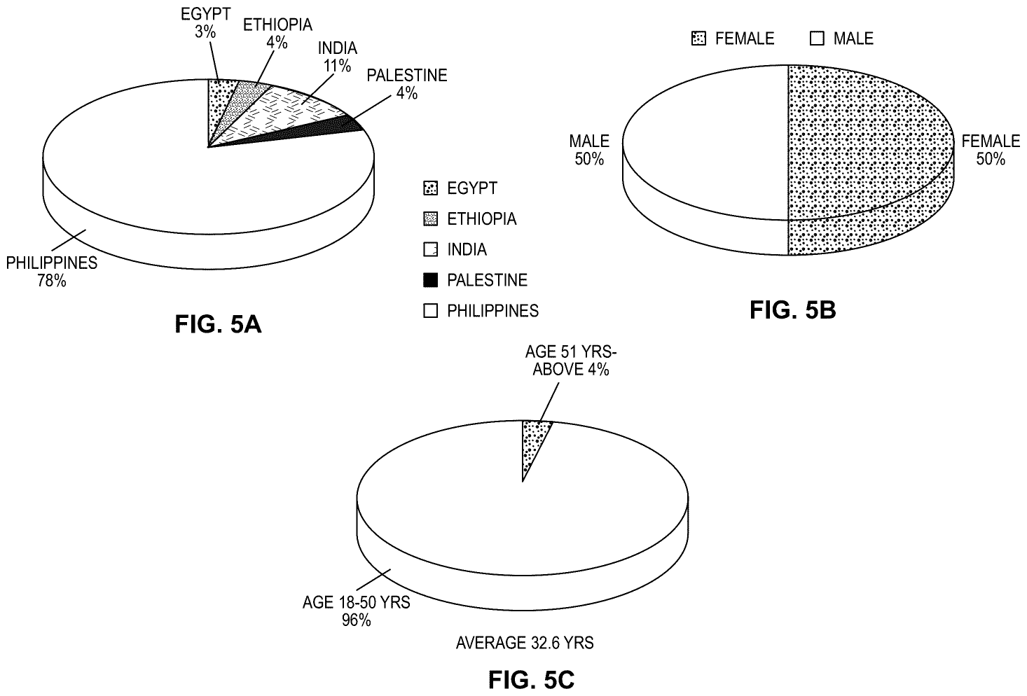

FIG. 5A illustrates the nationality distribution in the selected COVID-19 study group, according to some embodiments. FIG. 5B illustrates the gender distribution in the selected COVID-19 study group, according to some embodiments. FIG. 5C illustrates the age distribution in the selected COVID-19 study group, according to some embodiments. Participants with laboratory confirmed COVID-19 infection, as determined by a positive PCR, who agreed to participate, were screened within 72 hours after hotel admission to determine eligibility. PCR was collected after a minimum of 3 days from the symptom onset. However, those cases that had positive PCR for COVID-19 with less than 3 days of symptoms were also included. Eligible participants were randomized on Day 1 to the Composition A plus standard of care treatment group or to the placebo plus standard of care treatment group in a 1:1 ratio. Participants received treatment for up to 14 days or until the patient had negative a PCR test or discharged, whichever was earlier.

The Composition A plus standard of care treatment group received treatment with 3 g (30 mL) of Composition A syrup formulation three times daily (dose adjusted, if required) administered orally, for up to 14 days or until hospital discharge, whichever was earlier. The control group received standard of care treatment for COVID-19 pneumonia. All participants received treatment with low molecular weight heparin (LMWH) or unfractionated heparin (UFH) (appropriate dosing based upon participant status, renal function, and coagulopathies), unless already the patient already received oral or parenteral anticoagulants for an approved indication.

Table 3 shows the days to negative PCR/discharge between Composition A and the placebo. The mean of Composition A Group One minus the Placebo Group equals-2.07, with a 95% confidence interval of this difference: (−3.47 to −0.67). The mean comparison between the two groups revealed that the average days to negative PCR or discharge (recovery days) is less in the case of Composition A than the placebo. It was confirmed by the t-test that there is significant difference in the average number of days to negative PCR or discharge (recovery days) between Composition A and placebo groups. The two-tailed P value equals 0.0052, where the recovery days are significantly less in the case where the patient received Composition A.

TABLE 3
Days to Negative PCR/Discharge between
Composition A and Placebo.

| Group | Composition A Group | Placebo Group |
|---|---|---|
| Mean | 2.79 | 4.86 |
| SD | 1.67 | 1.92 |
| SEM | 0.45 | 0.51 |
| N | 14 | 14 |
| P Value | 0.0052 | |

Table 4 shows crosstabulation data for this example. The cross tabulation between vaccination and Composition A suggests that there are equal numbers of patients in the medication (Composition A) and Placebo groups (14 each). The majority of patients were exposed to Sino-pharm 2 doses (vaccination), 11 in each of the Composition A and Placebo groups.

TABLE 4
Medication Crosstabulation.
Vaccination * Trial Arm (Medication) Crosstabulation

| | | Trial Arm (Medication) | | |
|---|---|---|---|---|
| | | | Composition | |
| Count | | Placebo | A | Total |
| Vaccination | Not Vaccinated | 0 | 1 | 1 |
| | Pfizer 1 dose | 0 | 1 | 1 |
| | Sino-pharm 2 | 11 | 11 | 22 |
| | doses | | | |
| | Sino-pharm 3 | 2 | 0 | 2 |
| | doses | | | |
| | Sino-pharm 2 | 1 | 1 | 2 |
| | doses + Pfizer | | | |
| Total | | 14 | 14 | 28 |

Table 5 shows a chi-square test and p-values. A chi-square test determines if medication and vaccination are independent of each other. According to the table, the p-value is greater than 0.05 or 0.1 level, suggesting no significant association between vaccination and Composition A medication. In other words, both variables are independent of each other.

TABLE 5
Chi-Square Tests.
Chi-Square Tests

| | Value | df | p-value |
|---|---|---|---|
| Pearson Chi-Square | 4.000^{a} | 4 | 0.406 |
| Likelihood Ratio | 5.545 | 4 | 0.236 |
| Linear-by-Linear Association | 1.642 | 1 | 0.200 |
| N of Valid Cases | 28 | | |

^{a}8 cells (80.0%) have expected count less than 5. The minimum expected count is .50.

There were no deaths or SAEs reported during the study, and there were no AEs leading to discontinuation reported during the study. The study included 28 patients and results confirm a shorter duration of recovery in the treatment group with mean days to discharge of 2.78 days compared to 4.89 days for the placebo group, and a P value of 0.0052. This result shows the excellent effect and safety of Composition A for treatment of COVID-19, such as COVID-19 in stage II WHO.

### Example 3-Pharmaceutical Compositions for Treating Fibrosis and Cirrhosis

Liver cirrhosis remains one of the main causes of the morbidity and mortality in Sudan. A multi-center, open-labeled prospective cohort study was conducted in Khartoum, Sudan during the period from January 2018 to April 2021. Patients were enrolled from the hepatology out-patient clinics of four tertiary-level referral hospitals: Ibn Sina Specialized Hospital, Khartoum North Teaching Hospital, Police Hospital and Royal Care International Hospital. Targeted patients were all the adult Sudanese patients aged 18 years and above having evident liver fibrosis or cirrhosis, attended the hepatology out-patient clinics of any of the above mentioned hospitals, agreed to give consent for enrollment, and volunteered to receive Composition A for chronic liver disease (CLD). Patients with CLD due to hepatitis C (HCV) or hepatitis B (HBV) were enrolled only after completing antiviral therapy and had been off treatment for more than three months.

In contrast, patients were excluded from the study if they were younger than 18 years, had a body mass index of 40 kg/m² or above, were pregnant, had clinically evident ascitis, had an implantable cardiac device, had a confirmed diagnosis or history of malignancy, or those who were maintained on anti-viral therapy for Hepatitis B or C infection at the time of enrollment. Patients were also excluded if they had incomplete medical records or declined to provide consent for enrollment.

The diagnoses of liver fibrosis and liver cirrhosis were based on the diagnostic criteria set by practice guidelines for liver cirrhosis: the severity of CLD was determined non-invasively via elastography in patients with liver fibrosis using Fibroscan, which is a validated assessment method, with four stages. The normal range for a Fibroscan was determined as 2 to 7 Kilo Pascal (kPa); where scarring was reported at levels between 7-11 KPa. That stages were further classified into four stages as follows:
F0: no evidence scarring on Fibroscan.
F1: mild fibrosis, with 7-11 KPa on Fibroscan.
F2: moderate fibrosis with 12-19 KPa on Fibroscan.
F4: cirrhosis or advanced fibrosis with 20-75 KPa of Fibroscan.

Sixteen patients with liver fibrosis or cirrhosis that fulfilled the study inclusion and exclusion criteria volunteered for treatment with Composition A. The mean age of the study population was 50 years±14.8 (range 29-75 years). The majority of patients included were males (81.3%). Hepatitis B viral infection was the most common cause of CLD seen in 6 (46.2%) of participants, followed by schistosomiasis in 3 (18.8%), and cardiac cirrhosis in 2 (12.5%) patients. The mean duration of treatment for the study group was 4.5±4.2 months.

Enrolled volunteers were given 3 g (30 mL) of the Composition A liquid formulation, administered orally every 8 hours. At baseline, all patients were screened to confirm the absence of HCC (hepatocellular carcinoma) using abdominal ultrasonography and laboratory analyses, including Alpha Feto-protein levels. During the follow-up, all patients were seen at monthly intervals for up to 6 months and then at 3 month intervals. Periodic surveillance was performed with abdominal ultrasonography and laboratory analyses, including Alpha Feto-protein levels, every 3 to 6 months using screening tests for HCC and other complications of CLD. All patients had to undergo gastroscopy at time of enrollment and then every 1 to 2 years, as a screening test for gastroesophageal varices. Liver Fibroscan was done every 3 months together with the required and routine hematological and biochemical tests.

In this study, non-invasive elastography (Fibroscan) was used to assess regression of fibrosis/cirrhosis in patients. This method has been advocated by the NICE guidelines. Since severe acute exacerbation of hepatitis B can give less accurate results of Fibroscan, and to enhance the reliability of the results obtained, the results of liver elasticity scan were co-related to FIB-4 index results and showed comparable results. Changes from baseline in liver (type Fibroscan) between the two groups were compared by the paired t-test. Changes from baseline in serum markers for liver fibrosis FIB-4 (Fibroscan) for hepatitis B patients were compared using univariate and multiple regression analysis. The statistical significance is defined as follows:

Interim analysis when every 8 patients were enrolled and evaluated (effective new treatment if p<0.002) with futility look at p>0.830.
Second review was performed when 16 patients were enrolled and evaluated (effective treatment if p<0.014) with futility look at p>0.298.
The final analysis with the end of study period with P value for total enrolled patients (p<0.046).

All patients were adherent to therapy with Composition A given orally three times a day, and no dropouts were reported during the study period. Statistically significant changes were seen in the level of fibrosis when comparing the Fibroscan results before and after treatment (Table 6). When comparing the Fibroscan results obtained with the results obtained from the assessment of liver fibrosis by FIB-4 Index, there were no statistically significant changes regarding the percentage reduction in fibrosis between the two assessment methods (P 0.327). However, the impact of treatment duration was significant at 0.1 levels on the Fibroscan results Table 7. Minimal side effects of Composition A were reported during the study period. Importantly, administering the pharmaceutical composition resulted in regression of fibrosis and cirrhosis, as shown in Table 7.

TABLE 6
Fibroscan results for patients with chronic liver disease
(CLD) before and after treatment with Composition A.

| | | | | Treatment | Liver Scan (kpa) | | |
|---|---|---|---|---|---|---|---|
| | | | Age | Duration | Before | After | Percentage |
| ID | Cause | Gender | (Years) | (Month) | Treatment | Treatment | Reduction |
| 1 | Cardiac Cirrhosis | Male | 50 | 10 | 69.2 | 33.5 | 51.60% |
| 2 | Hepatitis B Virus | Female | 75 | 4 | 75 | 17.5 | 76.70% |
| 3 | Hepatitis B Virus | Male | 31 | 3 | 7.5 | 6.7 | 10.70% |
| 4 | Hepatitis B Virus | Male | 62 | 6 | 61.5 | 35.3 | 42.60% |
| 5 | Hepatitis B Virus | Male | 35 | 6 | 75 | 7.9 | 89.40% |
| 6 | Idiopathic | Female | 41 | 11 | 13.6 | 8.6 | 76.70% |
| | Cirrhosis | | | | | | |
| 7 | Cardiac Cirrhosis | Male | 34 | 2 | 51.4 | 34.2 | 33.50% |
| 8 | Hepatitis C Virus | Male | 70 | 1 | 7.4 | 6.4 | 13.50% |
| 9 | Hepatitis B Virus | Male | 34 | 3 | 33.3 | 5.8 | 82.60% |
| 10 | Hepatitis B Virus | Male | 52 | 1 | 11.8 | 9.5 | 19.50% |
| 11 | Alcoholic liver | Male | 60 | 2 | 32 | 23 | 28% |
| | cirrhosis | | | | | | |
| 12 | Sichistosomiasis | Male | 63 | 9 | 48 | 17.3 | 63% |
| 13 | Schistosomaisis | Male | 29 | 6 | 21.5 | 6.2 | 71% |
| 14 | Autoimmune | Male | 48 | 3 | 75 | 34.3 | 54% |
| | hepatitis | | | | | | |
| 15 | NASH* | Female | 51 | 3 | 18.9 | 11.8 | 37.60% |
| 16 | Schistosomaisis | Male | 65 | 2 | 75 | 48 | 60% |

*Non Alcoholic Steatohepatitis. P value 0.001

TABLE 7
Comparison of Fibroscan results with FIB-4 in patients
with chronic liver disease (CLD) using Composition A.

| | Liver Scan (kpa) | | | FIB-4 Index | | |
|---|---|---|---|---|---|---|
| | Before | After | Percentage | Before | After | Percentage |
| ID | Treatment | Treatment | Reduction | Treatment | Treatment | Reduction |
| 1 | 69.2 | 33.5 | 51.60% | 3.68 | 1.9 | 48.30% |
| 2 | 75 | 17.5 | 76.70% | 7.5 | 4.67 | 39% |
| 3 | 7.5 | 6.7 | 10.70% | 0.52 | 0.45 | 13% |
| 4 | 61.5 | 35.3 | 42.60% | 3.91 | 3.43 | 49% |
| 5 | 75 | 7.9 | 89.40% | 3.57 | 0.92 | 74.20% |
| 6 | 13.6 | 8.6 | 76.70% | 2.31 | 1.68 | 37.50% |
| 7 | 51.4 | 34.2 | 33.50% | 2.6 | 1.9 | 26.90% |
| 8 | 7.4 | 6.4 | 13.50% | 2.12 | 1.64 | 22.60% |
| 9 | 33.3 | 5.8 | 82.60% | 1.25 | 0.78 | 40% |
| 10 | 11.8 | 9.5 | 19.50% | 2.1 | 1.5 | 28.50% |
| 11 | 32 | 23 | 28% | 5.51 | 1.67 | 69.60% |
| 12 | 48 | 17.3 | 63% | 4.99 | 4.24 | 15% |
| 13 | 21.5 | 6.2 | 71% | 2.01 | 1.01 | 100% |
| 14 | 75 | 34.3 | 54% | 4.46 | 3.29 | 26% |
| 15 | 18.9 | 11.8 | 37.60% | 6.73 | 1.94 | 71% |
| 16 | 75 | 48 | 60% | 3.94 | 2.51 | 36% |

P value 0.327

With Composition A, reversal of fibrosis was achieved in only 6 months instead of the typical 5-7 years using antiviral treatment. Importantly, the duration of treatment for 70% of patients was less than 6 months, with a mean duration of 4.5±1.51 months. Furthermore, the patient with ID 5 in Tables 6-7 was referred for liver transplant a few months ahead of the trial and had remarkable regression of liver cirrhosis by 89.40% using Composition A, evident within a period of 6 months. A similar duration of therapy was required in those with a background of Hepatitis B virus infection. However, patients with CLD due to NASH and Schistosomaisis required a shorter duration of therapy. For example, only 2-3 months of treatment was needed to achieve remarkable results. The same applies to stages F2-F3, when only 2 months of treatment was needed in order to achieve significant recovery. The results of this trial have shown significant response to treatment with Composition A with a P value of less than 0.0001. When comparing the Fibroscan results to the Fibrosis-4 score before and after treatment, no significant differences were seen between the results, with a P value 0.327.

### Discussion of Clauses

Clause 1. A method of treating or reducing symptoms of coronavirus disease, the method comprising: administering a therapeutically effective amount of a pharmaceutical composition to a patient, the pharmaceutical composition including an extract of Balanites aegyptiaca, wherein the pharmaceutical composition is capable of treating or reducing symptoms of coronavirus disease.
Clause 2. The method of clause 1, wherein the coronavirus disease includes COVID-19 caused by severe acute respiratory syndrome coronavirus 2 (SARS-COV-2).
Clause 3. The method of clause 1, wherein the pharmaceutical composition further includes a pharmaceutical carrier, the Balanites aegyptiaca extract is a spray dried extract, and a weight/volume percentage of the spray dried extract in the pharmaceutical composition is greater than about 80 wt./vol %.
Clause 4. The method of clause 1, wherein the pharmaceutical composition further includes a pharmaceutical carrier, the Balanites aegyptiaca extract is a concentrated liquid extract, and a weight/volume percentage of the concentrated liquid extract in the pharmaceutical composition ranges from about 5 wt./vol % to about 60 wt./vol %.
Clause 5. The method of any one of the preceding clauses, wherein the Balanites aegyptiaca extract includes one or more of Quercetin, vanillic acid, syringic acid, 3-hydroxy-1-(4-hydroxy-3-methoxyphenyl)-1-propanone, furanocoumarin bergapten, and dihydrofuranocumarin D-marmesin.
Clause 6. The method of any one of the preceding clauses, wherein the therapeutically effective amount includes between about 150 mg and about 500 mg of Balanites aegyptiaca extract.
Clause 7. The method of any one of the preceding clauses, wherein the therapeutically effective amount includes between about 200 mg and about 500 mg of Balanites aegyptiaca extract.
Clause 8. The method of any one of the preceding clauses, wherein the pharmaceutical composition further includes one or more antimicrobial agents, wherein the one or more antimicrobial agents include at least one of methyl paraben, propyl paraben, and potassium sorbate.
Clause 9. The method of any one of the preceding clauses, wherein the pharmaceutical composition is capable of modulating cellular unfolded protein response (UPR) of viruses when administered.
Clause 10. The method of any one of the preceding clauses, wherein the pharmaceutical composition is capable of inactivating enveloped viruses and inhibiting virus proliferation when administered.
Clause 11. The method of clause 1, wherein the pharmaceutical composition further includes a pharmaceutical carrier and one or more antimicrobial agents, the Balanites aegyptiaca extract includes a spray dried extract, a weight/volume percentage of the spray dried extract in the pharmaceutical composition is greater than about 80 wt./vol %, and a weight/volume percentage of the one or more antimicrobial agents ranges from about 0.1 wt./vol % to about 10 wt./vol %.
Clause 12. The method of clause 1, wherein the pharmaceutical composition further includes a pharmaceutical carrier and one or more antimicrobial agents, the Balanites aegyptiaca extract includes a spray dried extract, a weight/volume percentage of the spray dried extract in the pharmaceutical composition is greater than about 86 wt./vol %, and a weight/volume percentage of the one or more antimicrobial agents ranges from about 0.1 wt./vol % to about 1 wt./vol %.
Clause 13. The method of clause 1, wherein the pharmaceutical composition further includes a pharmaceutical carrier and one or more antimicrobial agents, the Balanites aegyptiaca extract includes a concentrated liquid extract, a weight/volume percentage of the concentrated liquid extract in the pharmaceutical composition ranges from about 5 wt./vol % to about 20 wt./vol %, and a weight/volume percentage of the one or more antimicrobial agents ranges from about 0.1 wt./vol % to about 1 wt./vol %.
Clause 14. A method of treating or reducing symptoms of COVID-19, the method comprising: administering a therapeutically effective amount of an antiviral pharmaceutical composition to a patient, the antiviral pharmaceutical composition including a pharmaceutical carrier and an extract of Balanites aegyptiaca, wherein the antiviral pharmaceutical composition is capable of inhibiting severe acute respiratory syndrome coronavirus 2 (SARS-COV-2) proliferation.
Clause 15. The method of clause 14, wherein the antiviral pharmaceutical composition includes between about 200 mg and about 500 mg of Balanites aegyptiaca extract, and the antiviral pharmaceutical composition is capable of modulating cellular unfolded protein response (UPR) of viruses when the antiviral pharmaceutical composition is administered.
Clause 16. A pharmaceutical composition for treatment of coronavirus disease, the composition comprising: an extract of Balanites aegyptiaca including steroidal Saponins.
Clause 17. The pharmaceutical composition of clause 16 further including a pharmaceutical carrier, wherein the Balanites aegyptiaca extract is a spray dried extract, and wherein a weight/volume percentage of the spray dried extract in the composition is greater than about 80 wt./vol %.
Clause 18. The pharmaceutical composition of clause 16 further including a pharmaceutical carrier, wherein the Balanites aegyptiaca extract is a concentrated liquid extract, and wherein a weight/volume percentage of the concentrated liquid extract in the composition ranges from about 5 wt./vol % to about 60 wt./vol %.
Clause 19. The pharmaceutical composition of any one of the preceding clauses further including one or more antimicrobial agents, wherein the antimicrobial agents include at least one of methyl paraben, propyl paraben, and potassium sorbate.
Clause 20. The pharmaceutical composition of any one of the preceding clauses further including a viscosity increasing agent, wherein the viscosity increasing agent includes Acacia gum.
Clause 21. A method of treating fibrosis or cirrhosis, the method comprising: administering a therapeutically effective amount of a pharmaceutical composition to a patient, the pharmaceutical composition including a pharmaceutical carrier and an extract of Balanites aegyptiaca, wherein the pharmaceutical composition is capable of reducing at least one of fibrosis and cirrhosis when administered.
Clause 22. The method of clause 21, wherein the Balanites aegyptiaca extract is a spray dried extract, and the weight/volume percentage of the spray dried extract in the pharmaceutical composition is greater than about 80 wt./vol %.
Clause 23. The method of clause 21, wherein the Balanites aegyptiaca extract is a concentrated liquid extract, and the weight/volume percentage of the concentrated liquid extract in the pharmaceutical composition ranges from about 5 wt./vol % to about 60 wt./vol %.
Clause 24. The method of any one of clauses 21-23, wherein the pharmaceutical composition includes between about 200 mg and about 500 mg of Balanites aegyptiaca extract.
Clause 25. The method of any one of clauses 21-24, wherein the pharmaceutical composition further includes one or more antimicrobial agents, wherein the antimicrobial agents include at least one of methyl paraben, propyl paraben, and potassium sorbate.
Clause 26. The method of any one of clauses 21-25, wherein the pharmaceutical composition is capable of at least partially reversing one or more of liver fibrosis and liver cirrhosis when administered.
Clause 27. The method of any one of clauses 21-26, wherein the pharmaceutical composition is capable of reducing liver elasticity kPa values by over 30% when administered.
Clause 28. The method of any one of clauses 21-27, wherein the pharmaceutical composition is capable of reducing fat deposits in the liver when administered.
Clause 29. The method of any one of clauses 21-28, wherein the pharmaceutical composition is capable of regressing stage 4 liver fibrosis when administered.
Clause 30. The method of any one of clauses 21-29, wherein the pharmaceutical composition is capable of treating myocardial fibrosis when administered.
Clause 31. The method of any one of clauses 21-30, wherein the pharmaceutical composition is capable of exerting an antifibrotic effect when administered, by binding and regulating the function of a Lysyl oxidase like enzyme.
Clause 32. The method of any one of clauses 21-31, wherein the pharmaceutical composition is capable of reducing fibrosis when administered, through a calcium dependent mechanism by binding Ca+2.
Clause 33. The method of any one of clauses 21-32, wherein the pharmaceutical composition is capable of lowering serum lipids when administered.
Clause 34. The method of any one of clauses 21-33, wherein the pharmaceutical composition is capable of reversing fatty liver when administered.

While the disclosure has been described with reference to an exemplary embodiment(s), it will be understood by those skilled in the art that various changes may be made, and equivalents may be substituted for elements thereof without departing from the scope of the embodiment(s). In addition, many modifications may be made to adapt a particular situation or material to the teachings of the embodiment(s) without departing from the essential scope thereof. Therefore, it is intended that the disclosure is not limited to the disclosed embodiment(s), but that the disclosure will include all embodiments falling within the scope of the appended claims. Various examples have been described. These and other examples are within the scope of the following claims.

1. A method of treating or reducing symptoms of coronavirus disease, the method comprising:
administering a therapeutically effective amount of a pharmaceutical composition to a patient, the pharmaceutical composition including an extract of Balanites aegyptiaca, wherein the pharmaceutical composition is capable of treating or reducing symptoms of coronavirus disease.

2. The method of claim 1, wherein the coronavirus disease includes COVID-19 caused by severe acute respiratory syndrome coronavirus 2 (SARS-COV-2).

3. The method of claim 1, wherein the pharmaceutical composition further includes a pharmaceutical carrier, the Balanites aegyptiaca extract is a spray dried extract, and a weight/volume percentage of the spray dried extract in the pharmaceutical composition is greater than about 80 wt./vol %.

4. The method of claim 1, wherein the pharmaceutical composition further includes a pharmaceutical carrier, the Balanites aegyptiaca extract is a concentrated liquid extract, and a weight/volume percentage of the concentrated liquid extract in the pharmaceutical composition ranges from about 5 wt./vol % to about 60 wt./vol %.

5. The method of claim 1, wherein the Balanites aegyptiaca extract includes one or more of Quercetin, vanillic acid, syringic acid, 3-hydroxy-1-(4-hydroxy-3-methoxyphenyl)-1-propanone, furanocoumarin bergapten, and dihydrofuranocumarin D-marmesin.

6. The method of claim 1, wherein the therapeutically effective amount includes between about 150 mg and about 500 mg of Balanites aegyptiaca extract.

7. The method of claim 1, wherein the therapeutically effective amount includes between about 200 mg and about 500 mg of Balanites aegyptiaca extract.

8. The method of claim 1, wherein the pharmaceutical composition further includes one or more antimicrobial agents, wherein the one or more antimicrobial agents include at least one of methyl paraben, propyl paraben, and potassium sorbate.

9. The method of claim 1, wherein the pharmaceutical composition is capable of modulating cellular unfolded protein response (UPR) of viruses when administered.

10. The method of claim 1, wherein the pharmaceutical composition is capable of inactivating enveloped viruses and inhibiting virus proliferation when administered.

11. The method of claim 1, wherein the pharmaceutical composition further includes a pharmaceutical carrier and one or more antimicrobial agents, the Balanites aegyptiaca extract includes a spray dried extract, a weight/volume percentage of the spray dried extract in the pharmaceutical composition is greater than about 80 wt./vol %, and a weight/volume percentage of the one or more antimicrobial agents ranges from about 0.1 wt./vol % to about 10 wt./vol %.

12. The method of claim 1, wherein the pharmaceutical composition further includes a pharmaceutical carrier and one or more antimicrobial agents, the Balanites aegyptiaca extract includes a spray dried extract, a weight/volume percentage of the spray dried extract in the pharmaceutical composition is greater than about 86 wt./vol %, and a weight/volume percentage of the one or more antimicrobial agents ranges from about 0.1 wt./vol % to about 1 wt./vol %.

13. The method of claim 1, wherein the pharmaceutical composition further includes a pharmaceutical carrier and one or more antimicrobial agents, the Balanites aegyptiaca extract includes a concentrated liquid extract, a weight/volume percentage of the concentrated liquid extract in the pharmaceutical composition ranges from about 5 wt./vol % to about 20 wt./vol %, and a weight/volume percentage of the one or more antimicrobial agents ranges from about 0.1 wt./vol % to about 1 wt./vol %.

14. A method of treating or reducing symptoms of COVID-19, the method comprising:
administering a therapeutically effective amount of an antiviral pharmaceutical composition to a patient, the antiviral pharmaceutical composition including a pharmaceutical carrier and an extract of Balanites aegyptiaca, wherein the antiviral pharmaceutical composition is capable of inhibiting severe acute respiratory syndrome coronavirus 2 (SARS-COV-2) proliferation.

15. The method of claim 14, wherein the antiviral pharmaceutical composition includes between about 200 mg and about 500 mg of Balanites aegyptiaca extract, and the antiviral pharmaceutical composition is capable of modulating cellular unfolded protein response (UPR) of viruses when the antiviral pharmaceutical composition is administered.

16. A pharmaceutical composition for treatment of coronavirus disease, the composition comprising: an extract of Balanites aegyptiaca including steroidal Saponins.

17. The pharmaceutical composition of claim 16 further including a pharmaceutical carrier, wherein the Balanites aegyptiaca extract is a spray dried extract, and wherein a weight/volume percentage of the spray dried extract in the composition is greater than about 80 wt./vol %.

18. The pharmaceutical composition of claim 16 further including a pharmaceutical carrier, wherein the Balanites aegyptiaca extract is a concentrated liquid extract, and wherein a weight/volume percentage of the concentrated liquid extract in the composition ranges from about 5 wt./vol % to about 60 wt./vol %.

19. The pharmaceutical composition of claim 16 further including one or more antimicrobial agents, wherein the antimicrobial agents include at least one of methyl paraben, propyl paraben, and potassium sorbate.

20. The pharmaceutical composition of claim 16 further including a viscosity increasing agent, wherein the viscosity increasing agent includes Acacia gum.
